(19) United States
(12) Patent Application Publication
(10) Pub. No.: US 2015/0122265 A1
(43) Pub. Date: May 7, 2015

(54) Artificial Perforated Tarsorrhaphy

(71) Applicant: Nathan Horner, Los Angeles, CA (US)

(72) Inventor: Nathan Horner, Los Angeles, CA (US)

(21) Appl. No.: 14/264,009

(22) Filed: Apr. 28, 2014

Related U.S. Application Data
(63) Continuation of application No. 14/042,736, filed on Nov. 6, 2013, now abandoned.

(51) Int. Cl. A61F 9/04

(52) U.S. Cl. CPC A61F 9/045
USPC 128/845

(57) ABSTRACT

An Artificial Perforated Tarsorrhaphy is designed to hold an eyelid in a closed position to heal defects on the eye surface. The perforated mesh device includes a first edge, a second edge, an upper edge, a bottom edge, and an adhesive surface, wherein, the perforated mesh device is a non-rigid device. The device is adhered to the upper eyelid, wherein the perforated mesh material conforms to the contours of the eye ball, eye socket and eye brow, creating the function of holding down the upper eyelid and keeping the eye closed. The device is also designed so the corners of the eyes are exposed for comfort, and for adding medicine to the eye while the eyelid is in the closed position.

### CROSS REFERENCE

This application is related to U.S. application Ser. No. 14/042,736, filed Oct. 1, 2013, which is hereby incorporated by reference.

### FIELD OF INVENTION

The present invention relates to a device for aiding the healing of an eye after injury or a medical procedure. More specifically, the present invention relates to a device for holding an eyelid in a closed position, while allowing the eye to breath, and providing a means for inserting medicine into the closed eye.

### BACKGROUND

There has long been a clinical need tor a device to force an eyelid into a closed position, i.e., holding the upper eyelid down for healing alter various surgeries or injuries. Prior-art provides the painful and uncomfortable surgical procedure of sewing the eye closed called a Tarsorrhaphy. As illustrated in FIG. 1 (prior art), an upper eyelid is sewn together with the bottom eyelid to keep the eye closed during a healing period.

Other prior-art methods of holding the eye closed includes the standard cloth eye patch, as illustrated in FIG. 2 (prior-art), which smothers the eye, and medical tape, both being uncomfortable and are not effective for keeping the eye closed. Therefore, eye doctors commonly use a sewn Tarsorrhaphy as the standard method of keeping the eye closed, as illustrated in FIG. 1 (prior art).

Still other prior-art methods for holding an eyelid closed for eye healing has featured a substantially rigid eye splint, as disclosed in U.S. Pat. No. 6,034,293 to John F Stamler. The rigid eye splint has proved to be uncomfortable for the user wearing the device.

Accordingly, there is a needed for a device that heals defects on the eyes surface by painlessly, and comfortably holding the upper eye lid down, keeping the eye closed, making the eyelid its own natural bandage.

### BRIEF SUMMARY OF THE INVENTION

The Artificial Perforated Tarsorrhaphy device consist of a perforated mesh device for holding a facial eye in a closed position to heal defects on the eye surface. The perforated mesh device includes a first edge, a second edge, an upper edge, a bottom edge, and an adhesive surface, wherein, the perforated mesh device is a non-rigid device.

As the device is applied and adhered to the upper eyelid, the mesh material of the device conforms to the contours of the eye ball, eye socket and eye brow, creating the function of holding down the upper eyelid, keeping the eye closed. The device is also designed so the corners of the eyes are exposed for comfort, and for adding medicine to the eye while the eyelid is in the closed position.

It is contemplated that the perforated mesh device of the present invention is made of a 2-ply material from at least one of, a mesh fabric material; a hypoallergenic plastic material, or a latex free tape material.

### BRIEF DESCRIPTION OF THE DRAWINGS

FIG. 1 (prior art) Shows a front view photograph of actual Tarsorrhaphy procedure where the eye is sewn closed.

FIG. 2 (prior-art) Shows a standard cloth eye patch.

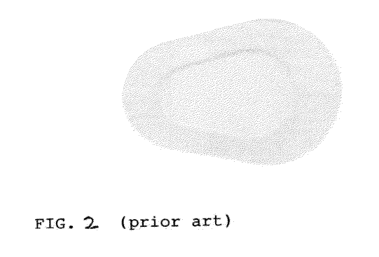

FIG. 3 Shows a photograph of the Artificial Perforated Tarsorrhaphy device of the present invention.

FIG. 4 Shows a photograph of the Artificial Perforated Tarsorrhaphy device of the present invention or a release liner.

FIG 5. Presents a drawing of the Artificial Perforated Tarsorrhaphy of the present invention.

FIG. 6 Shows a side view photograph of the Artificial Perforated Tarsorrhaphy of the present invention, to illustrate an approximate thickness of 2-ply material.

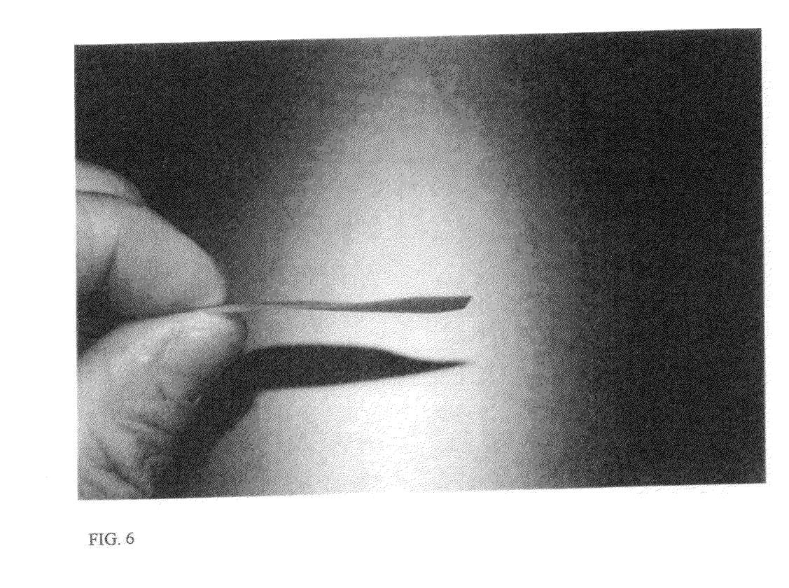

FIG. 7 Presents a profile view drawing of the applied Artificial Perforated Tarsorrhaphy of the present invention.

FIG. 8 Shows a front view photograph of the applied Artificial Perforated Tarsorrhaphy of the present invention.

FIG. 9 Illustrates a detailed profile view of the Artificial Perforated Tarsorrhaphy applied on the eye, displaying how the device functions to hold the eye lid down and how the device conforms to the eye socket, and under brow, leaving the corners of the eyes exposed.

FIG. 10 Shows a side profile photograph of Artificial Perforated Tarsorrhaphy applied close up on the eye.

FIG. 11 Illustrates a detail profile view of the Artificial Perforated Tarsorrhaphy applied close up on the eye, displaying how the upper material of the device rolls convexly around the under brow and forms to the eye socket and conforms to the radius of the eye

FIG. 12 Illustrates a front view of the applied device.

FIG. 13 Shows a front view photograph of the applied device

FIG. 14 Illustrates a front view drawing of Artificial Perforated Tarsorrhaphy applied on eye, and that the device is applied just above the opening of the eye on the upper eye lid.

FIG. 15 Shows a front view photograph of the Artificial Perforated Tarsorrhaphy applied on the eye.

### DETAILED DESCRIPTION AND BEST MODE OF IMPLEMENTATION

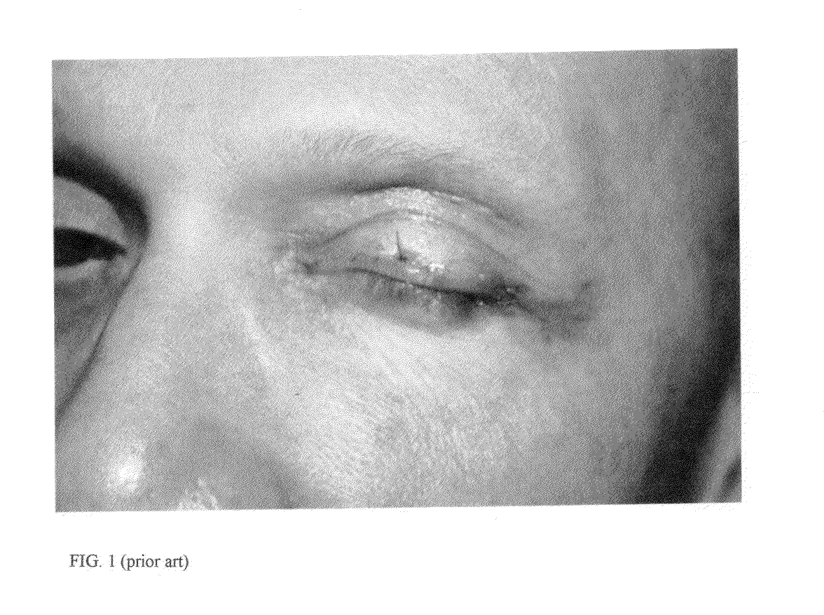

The Artificial Perforated Tarsorrhaphy heals defects on the eyes surface, as a painless option to the standard surgical procedure of sewing the eye closed called a Tarsorrhaphy, illustrated in FIG. 1 (prior art) by painlessly and comfortably holding the upper eyelid down, and holding the eye closed

The Artificial Perforated Tarsorrhaphy device of the present invention comprises improved procedures and articles for medically treating the facial eye after various surgeries and injuries, and for maintaining the health of the eye in the case of many other eye issues, including but not limited to nocturnal uses to hold the eye in a closed position in matters of dry eye, lazy eye, and strokes.

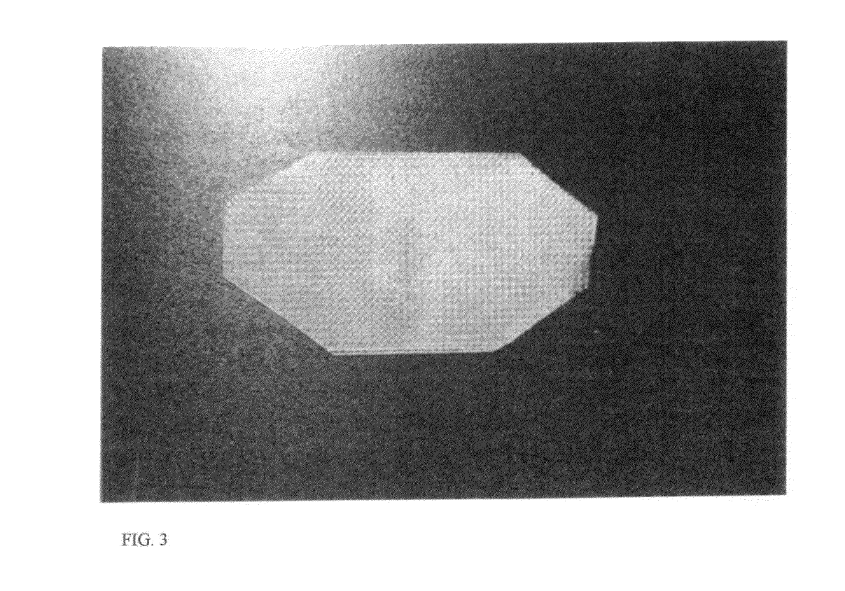

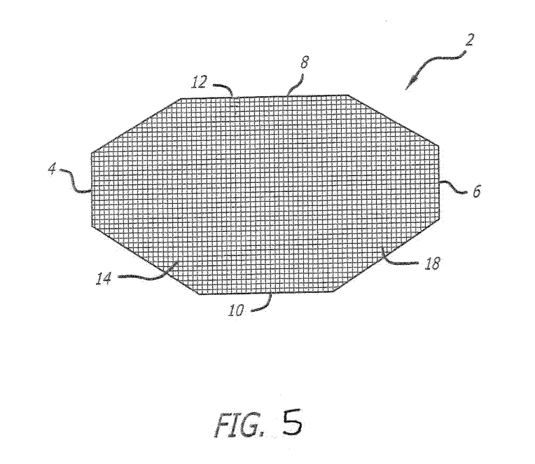

As illustrated in the accompanying photographs and drawings, the perforated mesh device of the present invention, designated by reference numeral 2-18 is designed for comfortably holding a facial eye m a closed position. The facial eye and other eye features are designated by reference numerals 20-38. Photographs and drawing consisting of FIGS. 3-5 display a preferred embodiment of the present invention. The mesh device 2 includes a first edge 4 for insertion into an eye socket 22 nose corner 24 of a user's face. A second edge 6 or distal edge, of the mesh device 2 of the present invention is opposite the first edge 4. The mesh device further includes an adhesive surface 12 (referenced to but not shown), a non-adhesive surface 14, an upper edge 8, and a bottom edge 10.

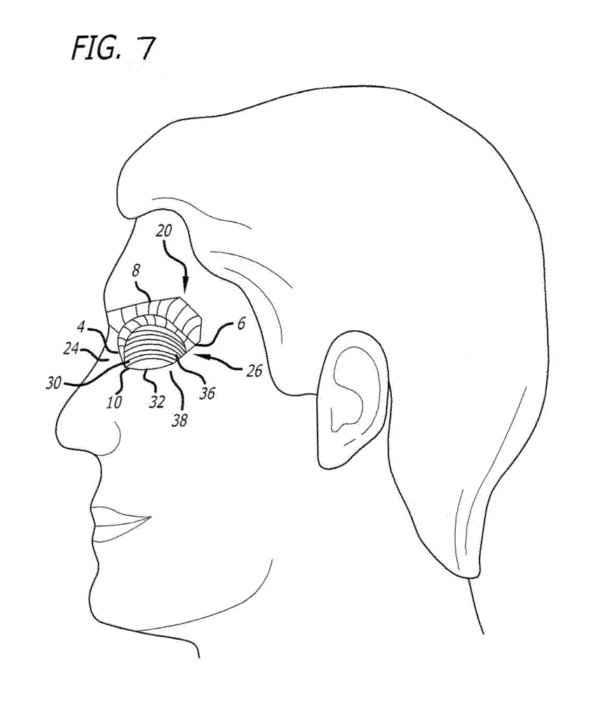

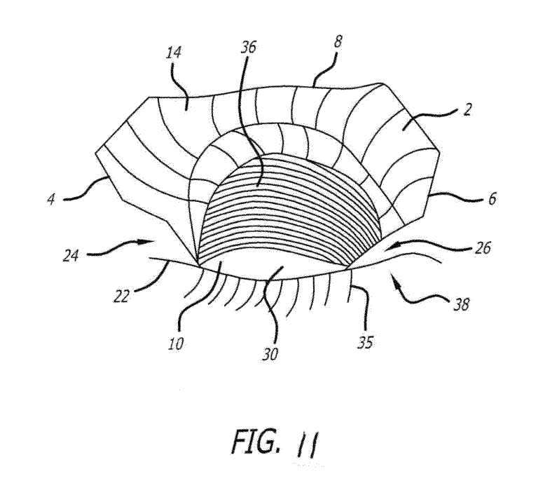

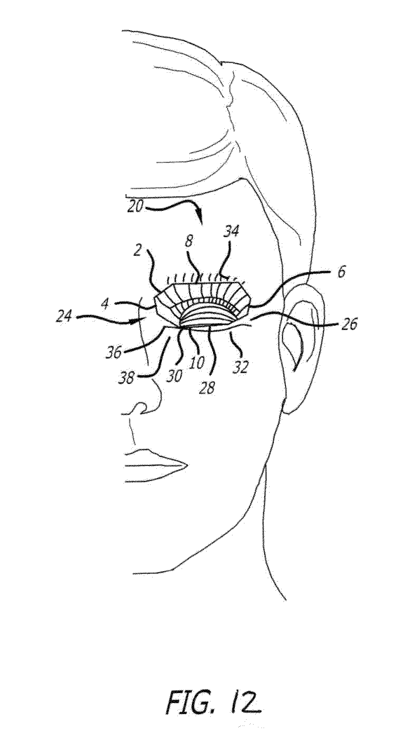

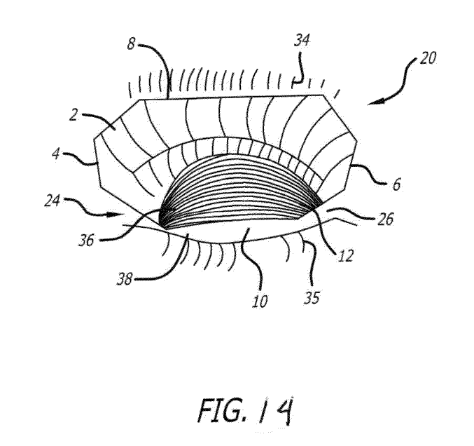

As illustrated in FIGS. 7-15, the upper edge 8 of the mesh device 2 is inserted onto the eye socket 22 below the eyebrow 34 and the mesh device bottom edge 10 is inserted onto the user's upper eyelid 30 above the bottom eyelid 32. The mesh device adhesive surface 12 adheres the mesh device 2 to the eye socket 22, and more specifically to the upper eyelid 30. The mesh device non-adhesive surface 14 does not contact the eye socket 22 nor the upper eyelid 30.

In the accompanying photographs and drawings the mesh device 2 is positioned on the user's left eye with first edge 4/nose corner edge 4 being placed on the left side of the user's face. However, it should be appreciated that the device is interchangeable for use on both the left and right sides of the user's face. When using the mesh device on the right eye, the reference numerals 4 and 6 may be interchanged to depict that the first edge/nose corner edge 4 of the present device is inserted onto the user's right facial area.

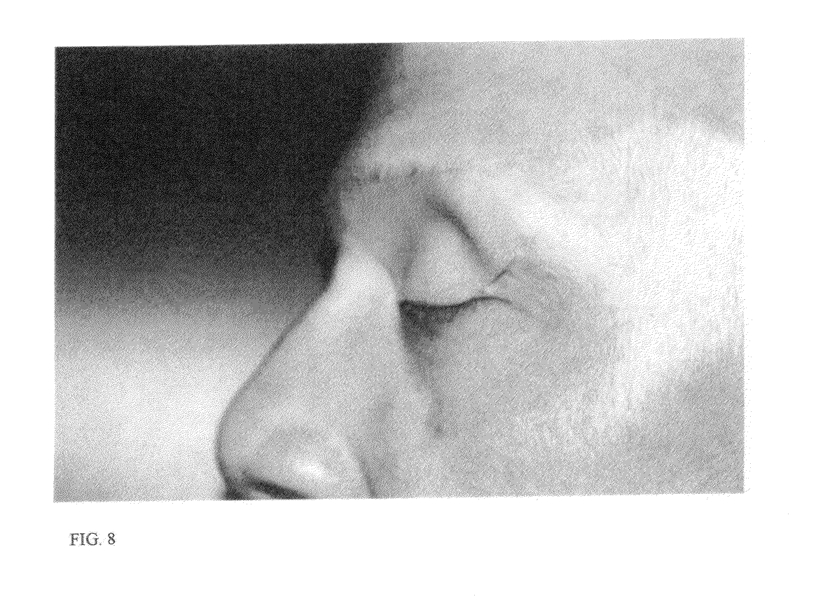

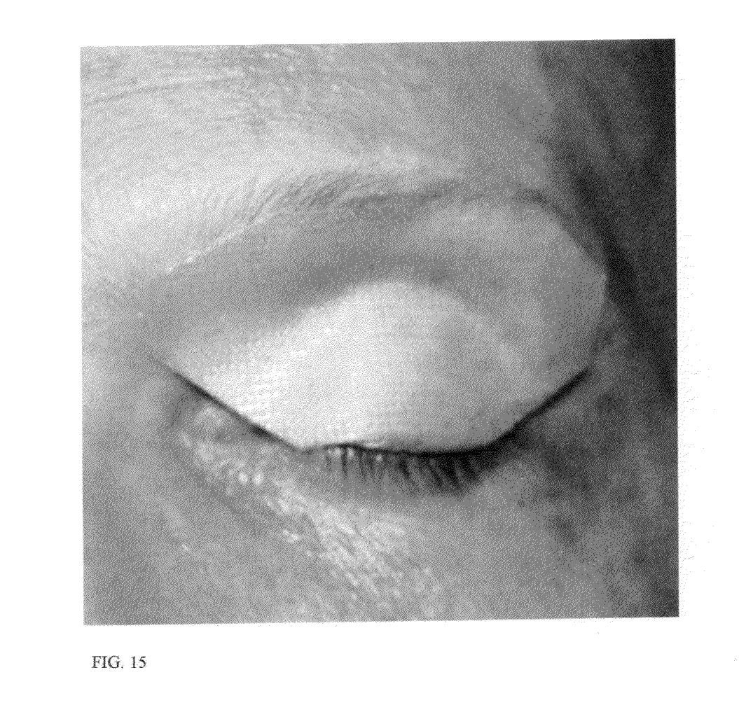

As shown in the photograph labeled FIG. 15, the mesh device becomes concave when conforming to the eye socket. This in turn curves the lower portion of the mesh material forcing the eyelid down end making the device act like the corrugation in steal, adding strength to the device, while achieving it's function to keep the eyelid in a closed position. The mesh device 2 upper material rolls convexly around the under brow. The mesh device 2 lower material hugs the contour of the eye ball 36, holding down the upper eyelid 30. See FIGS. 8, 9, 13, and 15.

The mesh device 2 may be made of two 1-ply material sheets of transpore plastic, hypoallergenic, adhesive, perforated, transparent, latex-free tape. Thou it would be molded as one piece, and could be made out of other perforated adhesive medical material. For example, the preferred embodiment of the present invention may be constructed from a 2-ply medical material of at least one of a mesh fabric material, a hypoallergenic plastic material, or a latex free tape material, all having an adhesive surface.

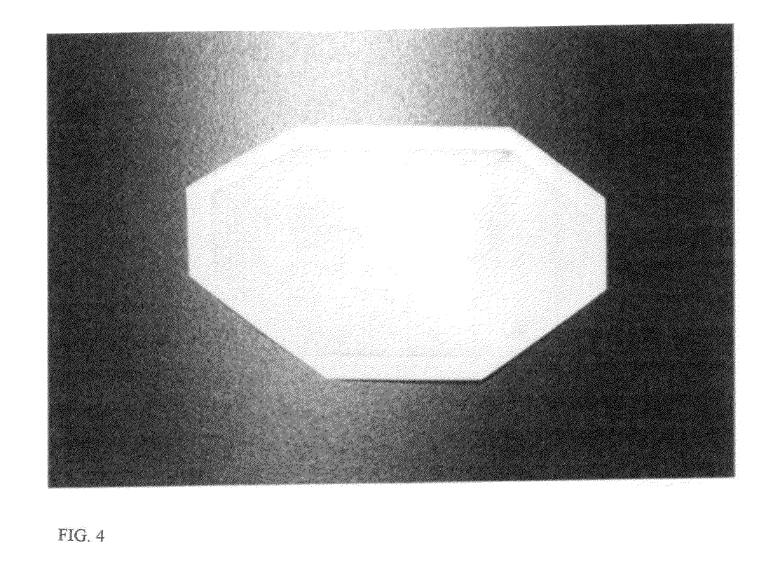

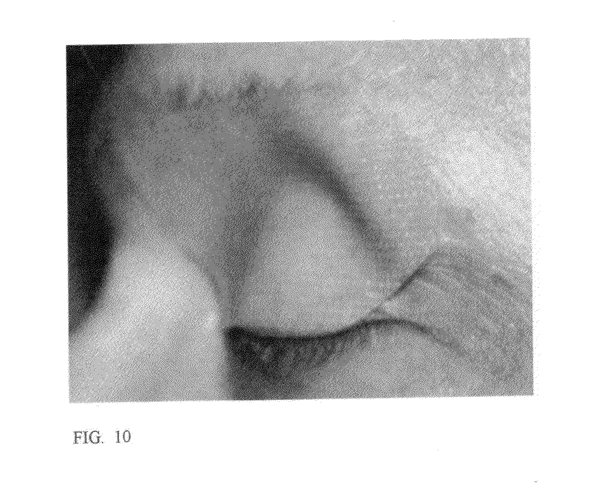

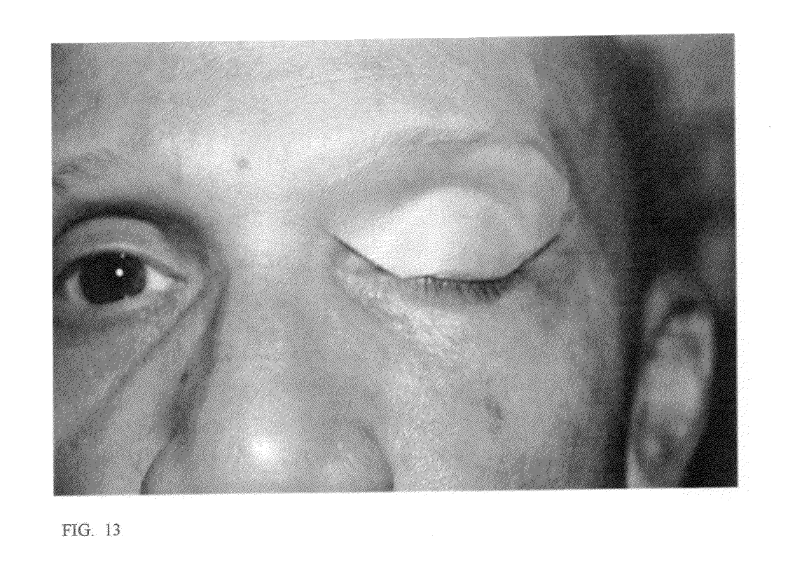

As shown in photographs FIG. 3, FIG. 4, FIG. 8, FIG. 10, FIG. 13, and FIG. 15, the mesh device 2 is designed with angles so the corners of the eyes are exposed for comfort, and to add medicine when the eyelid is closed. As illustrated in the FIG. 1A, FIG. 1B, FIG. 1D, and FIG. 3D, in an embodiment of the invention, the perforation or mesh of the thin material allows the user's eye to breathe.

In a preferred embodiment of the present invention, the mesh device 2 dimensions are 2.25″ length×1″ height×0135″ thickness Metric measurements may be 0.057150 m length×0.025400 m height×0.34 m thickness. However, it should be appreciated that the measurements and dimensions may vary to adapt to a specific user. The material cuts and tears easily for adjustments if needed. The adhesive holds strong, yet it allows the device to be easily removed.

In application of the mesh device 2, the user centers it over the closed upper eyelid, as shown in FIG. 13 and FIG. 15. The user then lines up the device bottom edge 10 just above the center of the opening of the eye on the upper eye lid above the lash. The user then gently rub the device 2 onto the eye lid, around the radius of the eye ball and up into the socket 20 and over the under part of the eyebrow 34 adhering the device 2 to the upper eyelid 30. This application holds down the eyelid, keeping the eye closed.

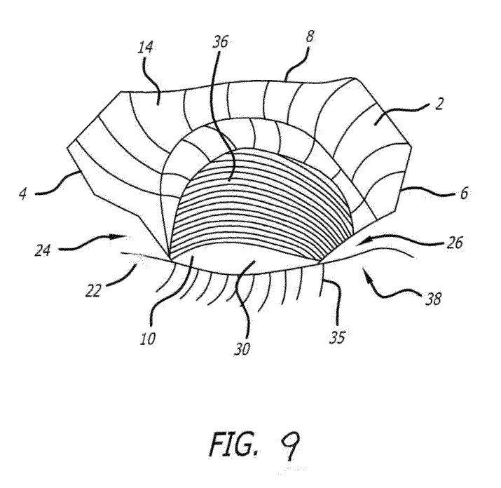

FIGS. 9, 11, 14 illustrate a detailed profile view of the Artificial Perforated Tarsorrhaphy device applied on the eye, displaying how the device function to hold the eyelid down, and how the device conforms to the eye socket and under brow, leaving the corners of the eye exposed

In the preferred embodiment of the present invention, the mesh device 2 will come packaged individually like a bandage, or Band-Aid™, which will even make it possible to be used in rural areas and Third World countries. It will be ideal for use by the military for treating eye injuries during combat. Each device is intended for one time use. It may be worn daily, but also can be worn at night while sleeping as in eases of chronic dry eye, lazy eye, and stroke. The device is affective to heal scratches and defects on the eyes surface, also to aid in healing after Lasik and or eye surgeries with a doctor's recommendation.

The mesh device 2 of the present invention is ideal for the elderly and children, and if necessary easily applied by a caregiver. It will be produced in various sizes for youths and adults for adaptation to all shapes and forms of eyes, due to varying facial characteristics and ethnicities. This device relieves unnecessary suffering.

The foregoing description of preferred embodiments is presented for purposes of illustration and description. Furthermore, the description is not intended to limit the invention to the form disclosed herein. Accordingly, variants and modifications consistent with the following teachings, and skill, and knowledge of the relevant art, are within the scope of the present invention.

I claim:

1. (canceled)

2. (canceled)

3. A device for holding an eye in a closed position, comprising:
a first edge;
a second edge;
an upper edge;
a bottom edge; and
an adhesive surface
wherein the device engages onto an upper eyelid of a facial eye, below an eye brow above the facial eye, and conforms to an eyeball contour formed on the upper eyelid when the upper eyelid is in a closed position; and
wherein the device upper edge rolls convexly under the eye brow and around an eye socket, and wherein the device bottom edge forms concavely around the eye socket, thereby strengthening the device in force the upper eyelid downward and holding the eye in the closed position.

4. The device of claim 4, wherein the device is a non-rigid device.

5. The device of claim 4, wherein the device adhesive surface is applied onto the upper eyelid when the eyelid is in the closed position.

6. The device of claim 4, wherein the device is designed for allowing a medication insertion into the facial eye when the upper eyelid is in the closed position.

7. The device of claim 4, wherein the device is made of a 2-ply material of at least one of a mesh fabric material, a hypoallergenic plastic material, and a latex free tape material.

8. The device of claim 7, wherein the device material is perforated.

9. A non-rigid device for holding a facial eye in a closed position, comprising:
a first edge;
a second edge;
an upper edge;
a bottom edge; and
an adhesive surface;
wherein the facial eye comprises, an eye socket having a nose corner and a distal corner; an eyeball; an upper eyelid; a bottom eyelid; and a eye brow above the eye socket;
wherein the non-rigid device engages onto the eye socket below the eye brow and conforms so an eyeball contour formed on the upper eyelid when the eyelid is in a closed position; and
a means for releasably holding the upper eyelid downward to the bottom eyelid in the closed position.

10. The non-rigid device of claim 9, wherein the means for releasably holding the upper eyelid downward to the bottom eyelid in the closed position comprises;
applying the non-rigid device adhesive surface onto the eyeball contour formed on the upper eyelid when the eyelid is in the closed position,
wherein the non-rigid device upper edge rolls convexly under the eye brow and around The eye socket, and wherein the non-rigid mesh device bottom edge forms concavely Around the eye socket, thereby strengthening the mesh device to force the upper eyelid downward and holding the eye in the closed position; and
disengaging the non-rigid device adhesive surface from the upper eyelid to release the eyelid from the closed position.

11. The non-rigid device of claim 9, wherein the device forms an opening at the eye socket nose edge and the eye socket distal corner for applying a medication to the eye, and for a user's comfort.

12. The non-rigid device of claim 11, wherein the device forms an octagonal shape for allowing the medication insertion in to the eye socket nose corner and the distal corner.

13. The non-rigid device of claim 10, therein the device allows the eye to breath in the closed position.

14. The non-rigid device of claim 10, wherein the device is made of a 2-ply sheet of at least one of a mesh fabric material, a hypoallergenic plastic material, and a latex free tape material.

15. The non-rigid device of claim 14, wherein the device further having an approximate dimension of 2.25 inches length, 1 inch height, and 0.135 inches thickness.

16. A perforated mesh device for holding a facial eye in a closed position, comprising:
a first edge;
a second edge;
an upper edge;
a bottom edge; and
an adhesive surface;
wherein the facial eye includes: an eye socket having a nose corner and a distal corner; an eyeball; an upper eyelid; a bottom eyelid; and an eye brow above the eye socket;
wherein the perforated mesh device engages onto the eye socket below the eye brow and conforms to an eyeball contour formed on the upper eyelid when the eyelid is in a closed position;
wherein the perforated mesh device is a non-rigid device, and
a means for releasably holding the upper eyelid downward to the bottom eyelid in the closed position comprising:
applying the perforated mesh device adhesive surface onto the eyeball contour formed on the upper eyelid when the eyelid is in closed position;
wherein the perforated mesh device upper edge rolls convexly under the eye brow and around the eye socket, and wherein the perforated mesh device bottom edge forms concavely around the eye socket, thereby strengthening the mesh device to force the upper eyelid downward and holding the eye in the closed position; and
disengaging the perforated mesh device adhesive surface from the upper eyelid to release the eyelid from the closed position

17. The perforated mesh device of claim 16, wherein the device is made of at least one of a mesh fabric material, a hypoallergenic plastic material, and a latex free tape material.

18. The perforated mesh device of claim 16, wherein the device is made of a 2-ply sheet of at least one of a mesh fabric material, a hypoallergenic plastic material, and a latex free tape material.

19. The perforated mesh device of claim 17, wherein the device further having an approximate dimension of 2.25 inches length, 1 inch height, and 0.135 inches thickness.

20. The perforated mesh device of claim 17, wherein the device forms an opening at the eye socket nose edge and the eye socket distal corner for applying a medication to the eye.

21. The perforated mesh device of claim 17, wherein the device allows the eye to breath in the closed position.
